(19) United States
(12) Patent Application Publication
(10) Pub. No.: US 2026/0232446 A1
(43) Pub. Date: Aug. 13, 2026

(54) Lattice Structure Device and Method

(71) Applicant: Orchid Orthopedic Solutions, LLC, Mason, MI (US)

(72) Inventors: Taban Larimian, Lansing, MI (US); Shilesh Jani, Collierville, TN (US); Scott Reese, Doylestown, PA (US); Philipp Kunz, Brittnau (CH); Steven Ostrowski, Whitmore Lake, MI (US)

(21) Appl. No.: 19/529,369

(22) Filed: Feb. 4, 2026

Related U.S. Application Data
(60) Provisional application No. 63/754,028, filed on Feb. 5, 2025.

(51) Int. Cl. A61F 2/30; A61F 2/38; B32B 3/10; B33Y 80/00

(52) U.S. Cl. CPC A61F 2/30771; A61F 2/3094; A61F 2/3859; B32B 3/10; A61F 2002/3092; A61F 2002/3093; A61F 2002/30985; B32B 2307/7376; B32B 2535/00; B33Y 80/00

(57) ABSTRACT

A device includes a substrate, a first lattice structure, and a second lattice structure. The first lattice structure is coupled to the substrate and defines a first plurality of pores. The second lattice structure is coupled to at least one of the substrate or the first lattice structure and at least partially disposed within the first plurality of pores. The second lattice structure defines a second plurality of pores in fluid communication with the first plurality of pores.

### CROSS-REFERENCE TO RELATED APPLICATIONS

This application claims the benefit of U.S. Provisional Application No. 63/754,028 filed Feb. 5, 2025, the entire disclosure of which is incorporated herein by reference.

### FIELD

The present disclosure relates to devices having one or more lattice structures and, more particularly, to medical implant devices having one or more lattice structures.

### BACKGROUND

For products and other devices (e.g., medical implant devices), surface characteristics play a critical role in the device's overall performance and, for example, integration with a body of a patient. A rough surface on an implant can promote enhanced cellular attachment and proliferation, which facilitates better osseointegration and tissue growth. This is especially important for implants made of materials such as titanium or other biocompatible metals, where a rough surface itself provides a scaffold for bone or tissue in-growth. By increasing the surface roughness, the implant can achieve a stronger bond with surrounding tissue, reducing the risk of implant failure and improving long-term stability. Additionally, rough surfaces may help to reduce the formation of fibrous tissue, allowing for a more natural, functional integration of the implant within the body.

Manufacturing implant devices with rough surfaces can be challenging due to the precision and complexity required in the production process. For example, achieving a uniformly rough surface with controlled roughness may be difficult to manage. Some implant materials, such as certain metals, ceramics, or polymers, may be difficult to work with when aiming to create rough surfaces. Additionally, manufacturing techniques for creating the rough surfaces can be expensive, time-consuming, and difficult to repeat. While known devices and manufacturing methods for adding rough surfaces have proven acceptable for their intended purpose, a continuous need for improvements remains in the pertinent art to address the challenges associated with manufacturing devices with rough surfaces.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A device includes a substrate, a first lattice structure, and a second lattice structure. The first lattice structure is coupled to the substrate and defines a first plurality of pores. The second lattice structure is coupled to at least one of the substrate or the first lattice structure and at least partially disposed within the first plurality of pores. The second lattice structure defines a second plurality of pores in fluid communication with the first plurality of pores.

A method of manufacturing a device includes forming a first lattice structure on a substrate. The first lattice structure defines a first plurality of pores. The method also includes forming a second lattice structure within the first plurality of pores. The second lattice structure defines a second plurality of pores in fluid communication with the first plurality of pores.

A device includes a substrate, a first lattice structure, and a second lattice structure. The first lattice structure is coupled to the substrate and includes a first plurality of struts defining a first outermost boundary offset from the substrate. The second lattice structure is coupled to at least one of the substrate or the first lattice structure and includes a second plurality of struts defining a second outermost boundary offset from the first outermost boundary.

An implant includes a substrate, a first lattice structure, and a second lattice structure. The substrate includes a proximal end and a distal end opposite the proximal end. The first lattice structure is coupled to the substrate between the proximal end and the distal end. The second lattice structure is intermingled with the first lattice structure between the proximal end and the distal end. At least one of the first lattice structure or the second lattice structure includes a plurality of struts defining an outermost boundary offset from the substrate. The outermost boundary defines a first roughness value at the distal end and a second roughness value that is less than the first roughness value at the proximal end.

An implant is produced by a process comprising providing a biocompatible substrate. The process also includes subjecting the substrate to a first additive manufacturing process to form a first lattice structure defining a first plurality of pores. The process further includes subjecting the substrate to a second additive manufacturing process to form a second lattice structure defining a second plurality of pores in fluid communication with the first plurality of pores.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a schematic view of an example device having one lattice structure in accordance with the principles of the present disclosure.

FIG. 2 is a schematic view of an example device having two lattice structures with different characteristics in accordance with the principles of the present disclosure.

FIG. 3 is a perspective view of an example device having two lattice structures with different strut sizes in accordance with the principles of the present disclosure.

FIG. 4 is a top view of an example device having two lattice structures with different pore sizes in accordance with the principles of the present disclosure.

FIG. 5 is a perspective view of an example device having two lattice structures with different thicknesses in accordance with the principles of the present disclosure.

FIG. 6 is a perspective view of an example device having two lattice structures with different porosities and pore sizes in accordance with the principles of the present disclosure.

FIG. 7 is a side view of the device of FIG. 6.

FIG. 8 is a perspective view of the device of FIG. 6 with one of the lattice structures hidden.

FIG. 9 is a perspective view of the device of FIG. 6 with the other one of lattice structures hidden.

FIG. 10 is a graph illustrating a ratio of a thickness of a second lattice structure to a thickness of a first lattice structure relative to the thickness of the first lattice structure in accordance with the principles of the present disclosure.

FIG. 11 a graph illustrating a thickness of a second lattice structure to a thickness of a first lattice structure in accordance with the principles of the present disclosure.

FIG. 12 is a side view of an example device having two lattice structures defining graded roughness in accordance with the principles of the present disclosure.

FIG. 13 is a side view of another example device having two lattice structures defining graded roughness in accordance with the principles of the present disclosure.

FIG. 14 is a side view of yet another example device having two lattice structures defining graded roughness in accordance with the principles of the present disclosure.

FIG. 15 is a side view of an additional example device having two lattice structures defining graded roughness in accordance with the principles of the present disclosure.

FIG. 16 is a side view of another example device having two lattice structures defining graded roughness in accordance with the principles of the present disclosure.

FIG. 17 is a side view of another example device having two lattice structures defining graded roughness in accordance with the principles of the present disclosure.

FIG. 18 is a side view of an example device having two lattice structures in accordance with the principles of the present disclosure.

FIG. 19 is a top view of an example device having three lattice structures with different characteristics in accordance with the principles of the present disclosure.

FIG. 20 is a side view of an example device having three lattice structures defining graded roughness in accordance with the principles of the present disclosure.

FIG. 21 is a side view of an example device having three overlapping stochastic structures in accordance with the principles of the present disclosure.

FIGS. 22-24 are side and perspective views of a hip stem implant device in accordance with the principles of the present disclosure.

FIG. 25 is a perspective view of a femoral knee implant device in accordance with the principles of the present disclosure.

FIG. 26 is a cross-sectional view of the femoral knee implant device of FIG. 25 taken along the line 26-26 of FIG. 25.

FIGS. 27 and 28 are front views of acetabular cup implant devices in accordance with the principles of the present disclosure.

FIG. 29 is a perspective view of a tibia base plate implant device in accordance with the principles of the present disclosure.

In the drawings, reference numbers may be reused to identify similar and/or identical elements.

### DETAILED DESCRIPTION

One aspect of the disclosure provides medical implant devices that are produced via additive manufacturing (AM). In various implementations, each of the devices includes one or more integrally printed lattice structures with certain desirable properties. The AM may be accomplished via electron beam melting (EBM), laser powder bed fusion (LPBF), binder jet (BJ), and stereolithography (SL), among others.

In various implementations, the devices include materials that can be produced by additive manufacturing (e.g., three-dimensionally printed). The materials may include metals, plastics, and ceramics, among others. For medical applications, the materials may include biocompatible titanium (and its alloys), stainless steel alloys, CoCr alloys, zirconium (and its alloys), tantalum (and its alloys), poly lactic acid, aluminum oxide, zirconium oxide, titanium carbide, and titanium nitride, among others.

In various implementations, a lattice structure means a portion of the device that is porous in nature (i.e., bereft of substantial material in the body of device). These pores may be open and interconnected, or they may be closed. Open means the pores are not occluded and therefore liquid and gases can flow in and out of the porous structure. Closed pores, on the other hand, are isolated from adjacent pores.

In various implementations, the basic ingredients of a porous lattice are such that surfaces with direct apposition to host tissue after implantation will allow the host bone tissue to grow or otherwise infiltrate the porous lattice.

In various implementations, characteristics of the one or more lattice structures are: (1) the porous volume should have a high degree of volumetric porosity, which directly results in high volumetric bone tissue infiltration, (2) the size of the pores interspersed within the porous volume should be sufficiently large to allow blood vessels to grow into and through them, (3) the thickness of the porous lattice body should be sufficient to allow for deep penetration of bone, and (4) the surface interface between host bone and porous lattice surface should be rough enough to resist significant micromotion, which can otherwise interfere with infiltration of blood vessels and bone tissue. These characteristics are designed in such a way that the porous structure is strong enough to withstand the high forces placed upon it during implantation surgery and subsequent load bearing by the patient during normal activities of daily living.

Another aspect of the disclosure relates to a method of manufacturing a porous lattice structure, where these different characteristics can be varied independent of each other. Additionally, these characteristics can be varied seamlessly across different portions of the implant. In various implementations, portions of the lattice structure are relatively smoother than other portions of the lattice structure, which is advantageous for ease of insertion during surgery, while other portions of the lattice structure are rough to facilitate rigid fixation.

While the devices are generally shown and described as being medical implants (e.g., for allowing bone to grow into a porous lattice structure), it will be appreciated that a device may be used in various other non-medical applications within the scope of the present disclosure.

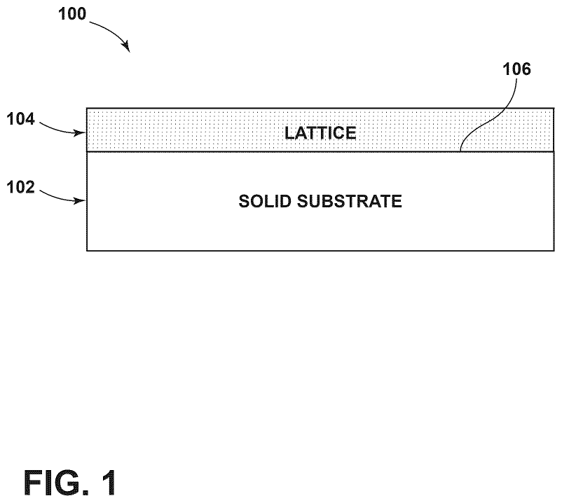

With reference to FIG. 1, an example device 100 is shown. The device 100 may comprise one or more of a variety of shapes, sizes, configurations, and/or materials. In various implementations, the device 100 includes a substrate 102 and a lattice structure 104 (e.g., a porous structure). The lattice structure 104 may be coupled to and extend from a surface 106 of the substrate 102. In various implementations, the substrate 102 is a solid substrate formed at least in part from a metal, a plastic, and/or a ceramic, among others. For medical applications, the substrate 102 may include biocompatible titanium (and/or its alloys), stainless steel alloys, CoCr alloys, zirconium (and/or its alloys), tantalum (and/or its alloys), polylactic acid, aluminum oxide, zirconium oxide, titanium carbide, and/or titanium nitride, among others. The lattice structure 104 may be formed at least in part from a metal, a plastic, and/or a ceramic, among others. For medical applications, the lattice structure 104 may include biocompatible titanium (and/or its alloys), stainless steel alloys, CoCr alloys, zirconium (and/or its alloys), tantalum (and/or its alloys), polylactic acid, aluminum oxide, zirconium oxide, titanium carbide, and/or titanium nitride, among others. As will be explained in more detail below, the device 100, including the substrate 102 and/or the lattice structure 104, may be formed using an additive manufacturing process such as electron beam melting (EBM), laser powder bed fusion (LPBF), binder jet (BJ), and stereolithography (SL), among others. In some implementations, the lattice structure 104 is integrally (e.g., monolithically) formed with the substrate 102 using an additive manufacturing process.

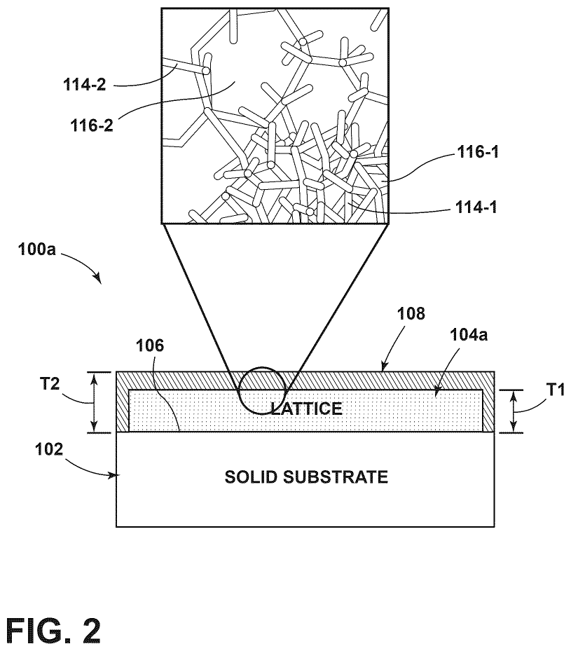

With reference to FIG. 2, an example device 100a is shown. In view of the similarity in structure and function of the device 100a to the device 100, like reference numerals are used hereinafter and in the drawings to identify like components while like reference numerals containing letter extensions (e.g., “a”) are used to identify those components that have been modified.

The device 100a may comprise one or more of a variety of shapes, sizes, configurations, and/or materials. In various implementations, the device 100a includes a substrate 102, a first lattice structure 104a, and a second lattice structure 108. The first and second lattice structures 104a, 108 may be coupled to, and extend from, a surface 106 of the substrate 102. The first and second lattice structures 104a, 108 may define an overlapping configuration. For example, the first and second lattice structures 104a, 108 may be intermingled. The first and second lattice structures 104a, 108 may cover a portion of the surface 106. Alternatively, the first and second lattice structures 104a, 108 may cover the entire surface 106.

In some example configurations, the first lattice structure 104a forms a base body and the second lattice structure 108 forms a roughness body. The first and second lattice structures 104a, 108 may have one or more different characteristics. As will be explained in more detail below, a characteristic may include porosity, pore size, strut size, lattice type, and/or thickness, among others.

In various implementations, the first and second lattice structures 104a, 108 have different lattice geometries. For example, one of the first or second lattice structures 104a, 108 may have a stochastic (e.g., random) structure and the other one of the first or second lattice structures 104a, 108 may have a unit cell (e.g., cubic, triply periodic minimal surface, or other repeating pattern) structure. In various implementations, the first lattice structure 104a defines a first thickness T1 and the second lattice structure 108 defines a second thickness T2. The second thickness T2 may be greater than the first thickness T1. The second thickness T2 may be less than the first thickness T1. In various implementations, the locations, the characteristics, and the geometries of the first and second lattice structures 104a, 108 are selectively chosen to provide the device 100a with the desired roughness on, e.g., the surface 106.

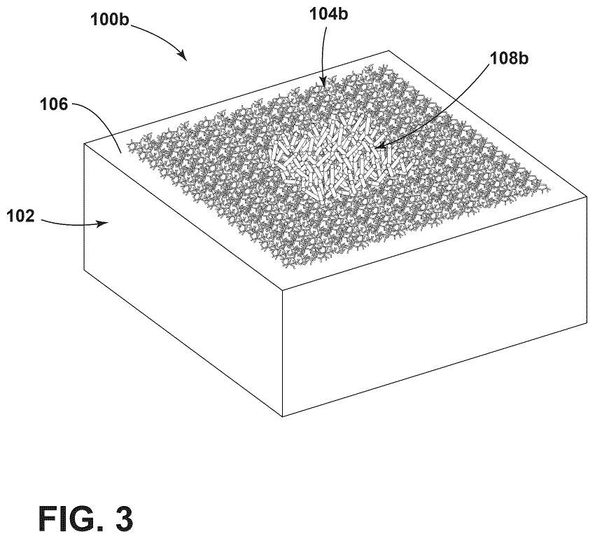

With reference to FIG. 3, an example device 100b is shown. In view of the similarity in structure and function of the device 100b to the devices 100, 100a, like reference numerals are used hereinafter and in the drawings to identify like components while like reference numerals containing letter extensions (e.g., “b”) are used to identify those components that have been modified.

The device 100b may comprise one or more of a variety of shapes, sizes, configurations, and/or materials. In various implementations, the device 100b includes a substrate 102, a first lattice structure 104b, and a second lattice structure 108b. The first and second lattice structures 104b, 108b may define an overlapping configuration. For example, the first and second lattice structures 104b, 108b may be intermingled. In various implementations, the first and second lattice structures 104b, 108b cover at least a portion of the surface 106. The second lattice structure 108b may be located proximate the center of the device 100b. Alternatively, the second lattice structure 108b may be located in a peripheral region of the device 100b.

In various implementations, the first lattice structure 104b may define a first strut size and the second lattice structure 108b may define a second strut size. The first strut size may be thicker than the second strut size. In various implementations, the first and second lattice structures 104b, 108b each have stochastic structures. The first and second lattice structures 104b, 108b may be located anywhere along the surface 106.

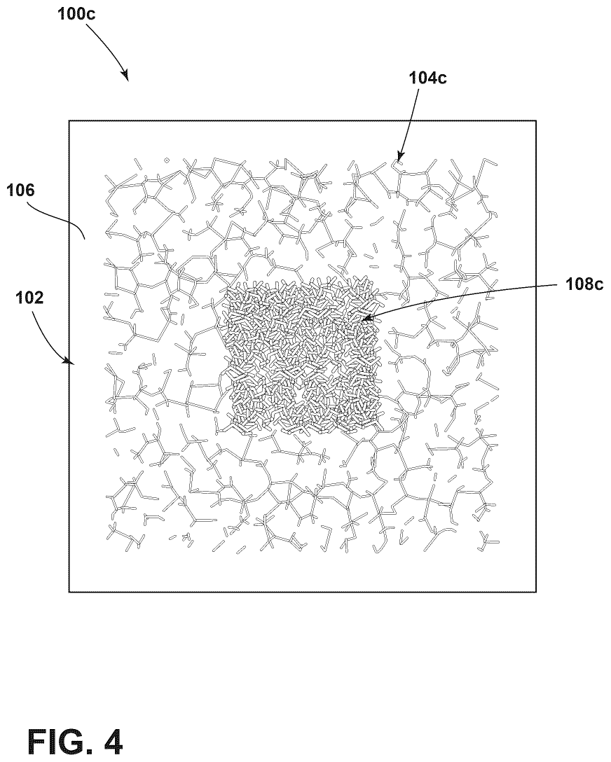

With reference to FIG. 4, an example device 100c is shown. In view of the similarity in structure and function of the device 100c to the devices 100-100b, like reference numerals are used hereinafter and in the drawings to identify like components while like reference numerals containing letter extensions (e.g., “c”) are used to identify those components that have been modified.

The device 100c may comprise one or more of a variety of shapes, sizes, configurations, and/or materials. In various implementations, the device 100c includes a substrate 102, a first lattice structure 104c, and a second lattice structure 108c. The first and second lattice structures 104c, 108c may define an overlapping configuration. For example, the first and second lattice structures 104c, 108c may be intermingled. In various implementations, the first and second lattice structures 104c, 108c cover at least a portion of the surface 106. The second lattice structure 108c may be located proximate the center of the device 100c. Alternatively, the second lattice structure 108b may be located in a peripheral region of the device 100c.

In various implementations, the first lattice structure 104c may define a first pore size and the second lattice structure 108c may define a second pore size. The first pore size may be larger than the second pore size. In various implementations, the first and second lattice structures 104c, 108c each have stochastic structures. Alternatively, one or both of the first or second lattice structures 104c, 108c may have unit cell or other structures. The first and second lattice structures 104c, 108c may be located anywhere along the surface 106.

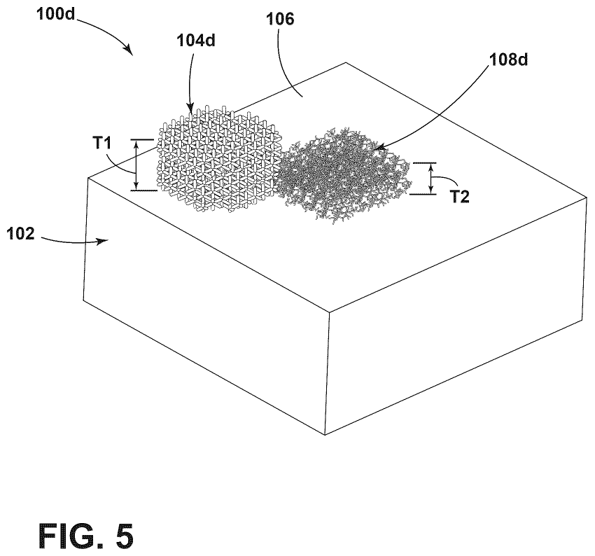

With reference to FIG. 5, an example device 100d is shown. In view of the similarity in structure and function of the device 100d to the devices 100-100c, like reference numerals are used hereinafter and in the drawings to identify like components while like reference numerals containing letter extensions (e.g., “d”) are used to identify those components that have been modified.

The device 100d may comprise one or more of a variety of shapes, sizes, configurations, and/or materials. In various implementations, the device 100d includes a substrate 102, a first lattice structure 104d, and a second lattice structure 108d. The second lattice structure 108d may be located proximate the center of the device 100d. The first lattice structure 104d may be disposed adjacent to the second lattice structure 108d. The first lattice structure 104d may define a unit cell structure. The second lattice structure 108d may define a stochastic structure. In various implementations, the first lattice structure 104d may define a first thickness T1 that is greater than a second thickness T2 of the second lattice structure 108d. The first and second lattice structures 104d, 108d may be located anywhere along the surface 106.

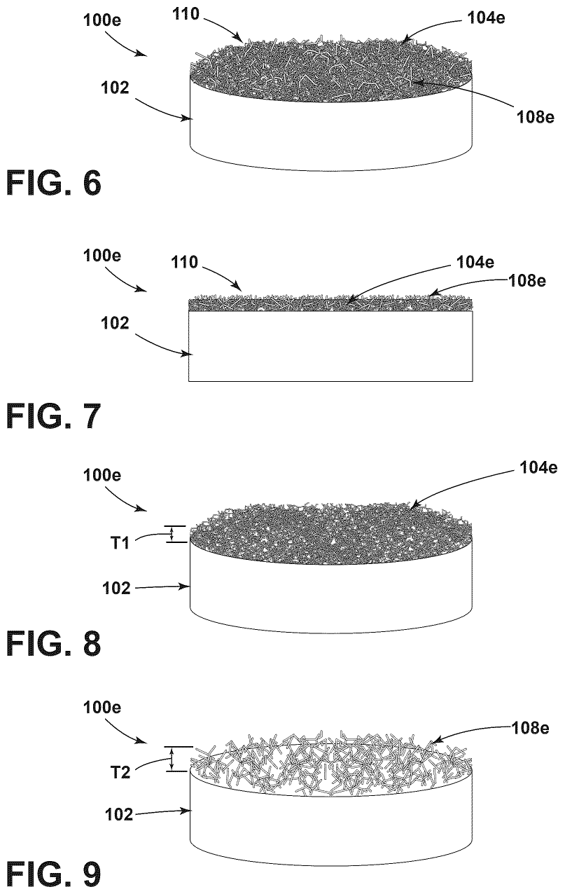

With reference to FIGS. 6-9, an example device 100e is shown. In view of the similarity in structure and function of the device 100e to the devices 100-100d, like reference numerals are used hereinafter and in the drawings to identify like components while like reference numerals containing letter extensions (e.g., “e”) are used to identify those components that have been modified.

The device 100e may comprise one or more of a variety of shapes, sizes, configurations, and/or materials. In various implementations, the device 100e includes a substrate 102, a first lattice structure 104e, and a second lattice structure 108e. The first and second lattice structures 104e, 108e may define an overlapping configuration. For example, the first and second lattice structures 104e, 108e may be intermingled. In various implementations, the first and second lattice structures 104e, 108e cover at least a portion of the surface 106. The first lattice structure 104e may define a base body and the second lattice structure 108e may define a roughness body.

The first lattice structure 104e may define a smaller pore size and a lower porosity in comparison with the second lattice structure 108e. In various implementations, the first lattice structure 104e may define a thickness T1 that is less than a thickness T2 of the second lattice structure 108e. In various implementations, the higher porosity and the larger pore size of the second lattice structure 108e provides the device 100e with a rough outer surface 110.

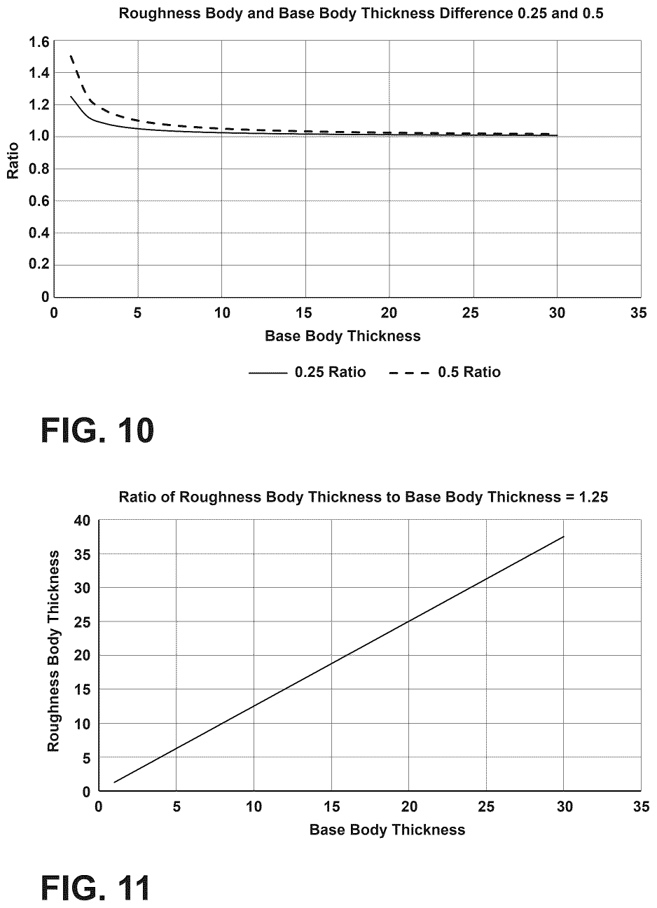

With reference to FIG. 10, a graph illustrating a ratio of a thickness T2 of a second lattice structure (e.g., the second lattice structure 108e) to a thickness T1 of a first lattice structure (e.g., the first lattice structure 104e) relative to the thickness T1 of the first lattice structure is shown. When the difference between the thickness T1 of the first lattice structure (e.g., the first lattice structure 104e) and the thickness T2 of the second lattice structure (e.g., the second lattice structure 108e) is maintained at, e.g., 0.25 mm or 0.5 mm, it can be seen that starting at a thickness T1 of approximately 1 millimeter, as the thickness T1 increases, the ratio of the thickness T2 to the thickness T1 approaches a value of one asymptotically.

With reference to FIG. 11, a graph illustrating a thickness T2 of a second lattice structure (e.g., the second lattice structure 108e) relative to a thickness T1 of a first lattice structure (e.g., the first lattice structure 104e) is shown. When the ratio of the thickness T2 to the thickness T1 is maintained at approximately (e.g., ±10%) 1.25, the relationship between the thickness T2 and the thickness T1 is linear.

As shown, the thickness (e.g., T2) of the roughness body (e.g., the second lattice structure 108e) and the thickness (e.g., T1) of the base body (e.g., the first lattice structure 104e) may be varied linearly to obtain the desired outer surface 110 roughness of the device. Alternatively, the thickness of the roughness body and the thickness of the base body may be varied in a non-linear fashion.

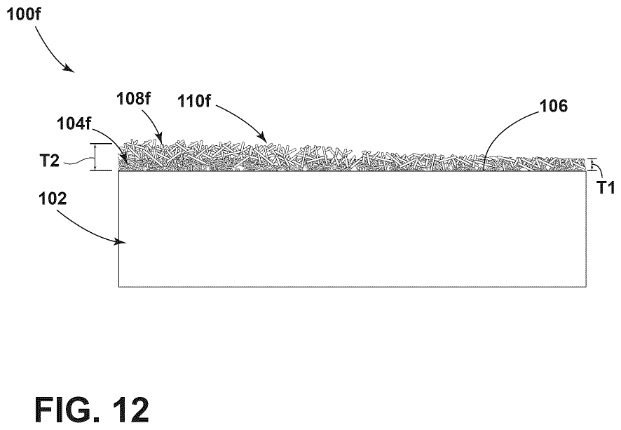

With reference to FIG. 12, an example device 100f is shown. In view of the similarity in structure and function of the device 100f to the devices 100-100e, like reference numerals are used hereinafter and in the drawings to identify like components while like reference numerals containing letter extensions (e.g., “f”) are used to identify those components that have been modified.

The device 100f may comprise one or more of a variety of shapes, sizes, configurations, and/or materials. In various implementations, the device 100f includes a substrate 102, a first lattice structure 104f, and a second lattice structure 108f. The first lattice structure 104f may define a base body and the second lattice structure 108f may define a roughness body. The first and second lattice structures 104f, 108f may each define a stochastic structure. In various implementations, the thickness T1 of the first lattice structure 104f is constant (e.g., the same) along the surface 106. In various implementations, the thickness T2 of the second lattice structure 108f gradually increases along the surface 106. In various implementations, increasing the thickness T2 of the second lattice structure 108f increases the roughness of the outer surface 110f of the device 100f. In this case, the overall thickness of the second lattice structure 108f and the first lattice structure 104f, which may be equal to the thickness T2 of the second lattice structure 108f, increases from the thickness T1 to a maximum progressing going to the left relative to FIG. 12 such that (i) the roughness increases going right to left relative to FIG. 12 and (ii) in an implant, the press fit increases going right to left relative to FIG. 12.

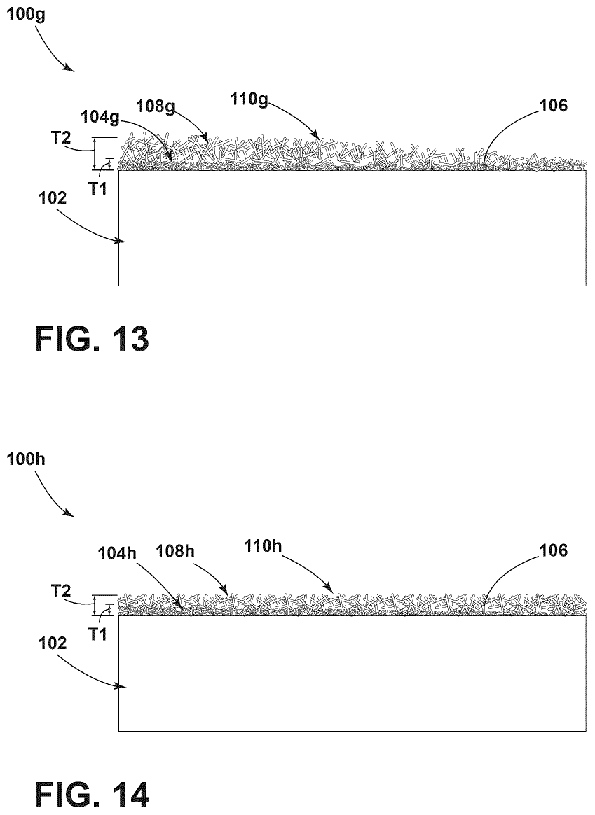

With reference to FIG. 13, an example device 100g is shown. In view of the similarity in structure and function of the device 100g to the devices 100-100f, like reference numerals are used hereinafter and in the drawings to identify like components while like reference numerals containing letter extensions (e.g., “g”) are used to identify those components that have been modified.

The device 100g may comprise one or more of a variety of shapes, sizes, configurations, and/or materials. In various implementations, the device 100g includes a substrate 102, a first lattice structure 104g, and a second lattice structure 108g. The first lattice structure 104g may define a base body and the second lattice structure 108g may define a roughness body. The first and second lattice structures 104f, 108f may each define a stochastic structure. In various implementations, the thickness T1 of the first lattice structure 104f is constant (e.g., the same) along the surface 106. In various implementations, the second lattice structure 108g defines a wedge-shaped configuration. For example, the thickness T2 of the second lattice structure 108f gradually increases along the surface 106. The wedge-shaped configuration increases the roughness of the outer surface 110g while providing a uniform transition between sections of the device 100g.

With reference to FIG. 14, an example device 100h is shown. In view of the similarity in structure and function of the device 100h to the devices 100-100g, like reference numerals are used hereinafter and in the drawings to identify like components while like reference numerals containing letter extensions (e.g., “h”) are used to identify those components that have been modified.

The device 100h may comprise one or more of a variety of shapes, sizes, configurations, and/or materials. In various implementations, the device 100h includes a substrate 102, a first lattice structure 104h, and a second lattice structure 108h. The first lattice structure 104h may define a base body and the second lattice structure 108h may define a roughness body. The first and second lattice structures 104h, 108h may each define a stochastic structure. In various implementations, the thickness T2 of the second lattice structure 108h is constant (e.g., the same) along the surface 106. In various implementations, the thickness T1 of the first lattice structure 104h gradually decreases along the surface 106. In various implementations, decreasing the thickness T1 of the first lattice structure 104h increases the roughness of the outer surface 110h of the device 100h.

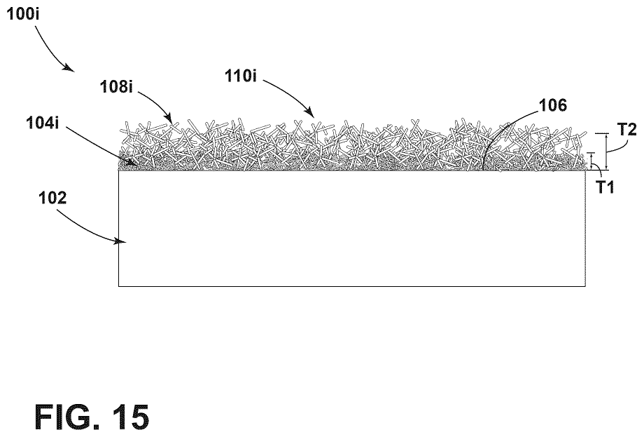

With reference to FIG. 15, an example device 100i is shown. In view of the similarity in structure and function of the device 100i to the devices 100-100h, like reference numerals are used hereinafter and in the drawings to identify like components while like reference numerals containing letter extensions (e.g., “i”) are used to identify those components that have been modified.

The device 100i may comprise one or more of a variety of shapes, sizes, configurations, and/or materials. In various implementations, the device 100i includes a substrate 102, a first lattice structure 104i, and a second lattice structure 108i. The first lattice structure 104i may define a base body and the second lattice structure 108i may define a roughness body. The first and second lattice structures 104i, 108i may each define a stochastic structure. In various implementations, the thickness T2 of the second lattice structure 108i is constant (e.g., the same) along the surface 106. In various implementations, the first lattice structure 104i defines a wedge-shaped configuration. For example, the thickness T1 of the first lattice structure 108i gradually decreases along the surface 106. The wedge shape configuration increases the roughness of the outer surface 110i while providing a uniform transition between sections of the device 100i.

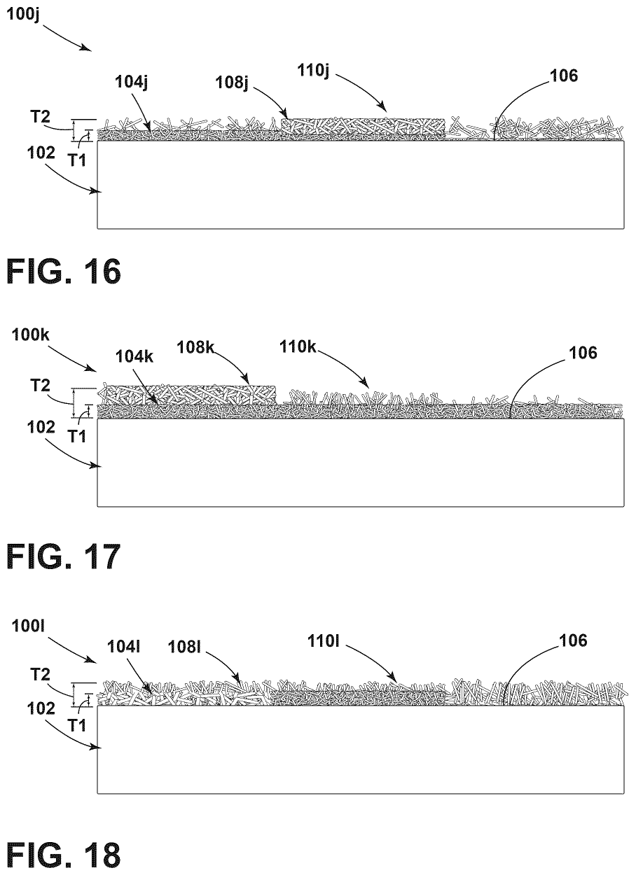

With reference to FIG. 16, an example device 100j is shown. In view of the similarity in structure and function of the device 100j to the devices 100-100i, like reference numerals are used hereinafter and in the drawings to identify like components while like reference numerals containing letter extensions (e.g., “j”) are used to identify those components that have been modified.

The device 100j may comprise one or more of a variety of shapes, sizes, configurations, and/or materials. In various implementations, the device 100j includes a substrate 102, a first lattice structure 104j, and a second lattice structure 108j. The first lattice structure 104j may define a base body and the second lattice structure 108j may define a roughness body. In various implementations, the thickness T2 of the second lattice structure 108j is constant (e.g., the same), while the pore size and the porosity of the second lattice structure 108j varies along the surface 106. In various implementations, the thickness T1 of the first lattice structure 104j varies along the surface 106. The foregoing characteristics of the first and second lattice structures 104j, 108j provide the outer surface 110j of the device 100j with a roughness that varies. For example, the roughness of the surface 110j may be graded from a first side of the device 100j to a second side of the device 100j that is opposite the first side of the device 100j.

With reference to FIG. 17, an example device 100k is shown. In view of the similarity in structure and function of the device 100k to the devices 100-100j, like reference numerals are used hereinafter and in the drawings to identify like components while like reference numerals containing letter extensions (e.g., “k”) are used to identify those components that have been modified.

The device 100k may comprise one or more of a variety of shapes, sizes, configurations, and/or materials. In various implementations, the device 100k includes a substrate 102, a first lattice structure 104k, and a second lattice structure 108k. The first lattice structure 104k may define a base body and the second lattice structure 108k may define a roughness body. In various implementations, the thickness T1 of the first lattice structure 104j is constant (e.g., the same) along the surface 106. In various implementations, the thickness T2, the pore size, and the porosity of the second lattice structure 108j varies along the surface 106. The foregoing characteristics of the first and second lattice structures 104k, 108k provide the outer surface 110k of the device 100k with a roughness that varies. For example, the roughness of the surface 110k may be graded from a first side of the device 100k to a second side of the device 100k that is opposite the first side of the device 100k.

With reference to FIG. 18, an example device 100l is shown. In view of the similarity in structure and function of the device 100l to the devices 100-100k, like reference numerals are used hereinafter and in the drawings to identify like components while like reference numerals containing letter extensions (e.g., “l”) are used to identify those components that have been modified.

The device 100l may comprise one or more of a variety of shapes, sizes, configurations, and/or materials. In various implementations, the device 100l includes a substrate 102, a first lattice structure 104l, and a second lattice structure 108l. The first lattice structure 104l may define a base body and the second lattice structure 108l may define a roughness body. In various implementations, the thickness T1, the pore size, and the strut size of the first lattice structure 104l and/or the second lattice structure 108l varies along the surface 106. In various implementations, the thickness T2 of the second lattice structure 108l is constant (e.g., the same) along the surface 106. The foregoing characteristics of the first and second lattice structures 104l, 108l provide the outer surface 110l of the device 100l with constant roughness, while the porosity percentage is varied in different sections of the device 100l dependent on the pore and strut sizes of the first lattice structure 104l.

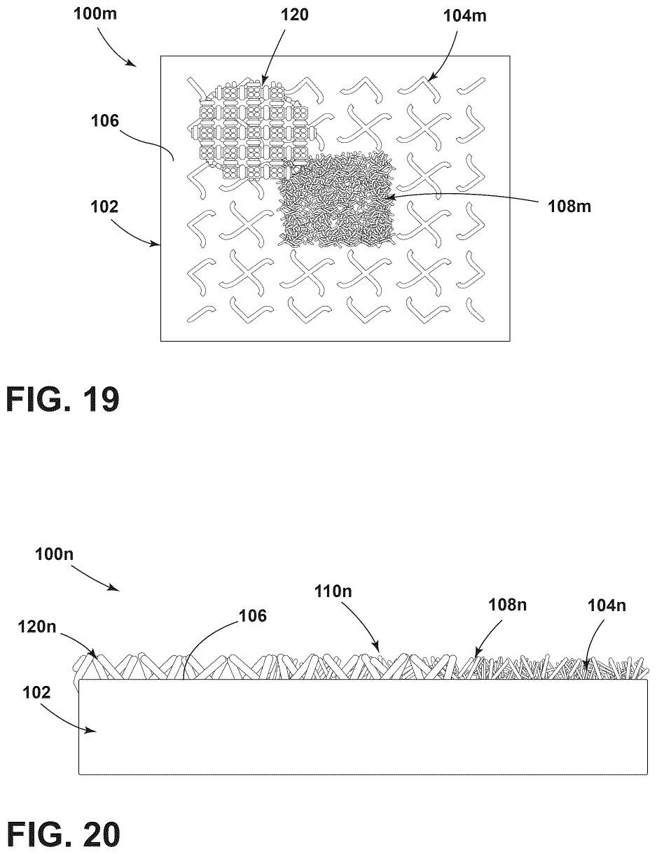

With reference to FIG. 19, an example device 100m is shown. In view of the similarity in structure and function of the device 100m to the devices 100-100l, like reference numerals are used hereinafter and in the drawings to identify like components while like reference numerals containing letter extensions (e.g., “m”) are used to identify those components that have been modified.

The device 100m may comprise one or more of a variety of shapes, sizes, configurations, and/or materials. In various implementations, the device 100m includes a substrate 102, a first lattice structure 104m, a second lattice structure 108m, and a third lattice structure 120. The first, second, and third lattice structures 104m, 108m, 120 may be coupled to and extend from a surface 106 of the substrate 102. The first and third lattice structures 104m, 120 may define unit cell structures and the second lattice structure 108m may define a stochastic structure. In other implementations the first, second, and third lattice structures 104m, 108m, 120 may have stochastic or unit cell structures, in any combination. At least portions of the first, second, and third lattice structures 104m, 108m, 120 may overlap. The first, second, and third lattice structures 104m, 108m, 120 may not overlap. The first, second, and third lattice structures 104m, 108m, 120 may disposed anywhere along the surface 106.

With reference to FIG. 20, an example device 100n is shown. In view of the similarity in structure and function of the device 100n to the devices 100-100m, like reference numerals are used hereinafter and in the drawings to identify like components while like reference numerals containing letter extensions (e.g., “n”) are used to identify those components that have been modified.

The device 100n may comprise one or more of a variety of shapes, sizes, configurations, and/or materials. In various implementations, the device 100n includes a substrate 102, a first lattice structure 104n, a second lattice structure 108n, and a third lattice structure 120n. The first and lattice structures 104n, 108n may define stochastic structures. The third lattice structure 120n may define a unit cell structure. In various implementations, the outer surface 110n changes from fully stochastic to a combination of stochastic and unit cell and to fully unit cell. The foregoing characteristics of the first, second, and third lattice structures 104n, 108n, 120n provide the outer surface 110n of the device 100n with varying or graded roughness.

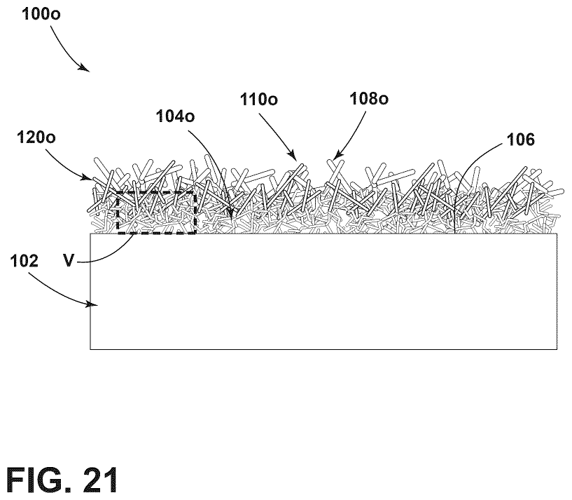

With reference to FIG. 21, an example device 100o is shown. In view of the similarity in structure and function of the device 100o to the devices 100-100n, like reference numerals are used hereinafter and in the drawings to identify like components while like reference numerals containing letter extensions (e.g., “o”) are used to identify those components that have been modified.

The device 100o may comprise one or more of a variety of shapes, sizes, configurations, and/or materials. In various implementations, the device 100o includes a substrate 102, a first lattice structure 104o, a second lattice structure 108o, and a third lattice structure 120o. In various implementations, the first, second, and third lattice structures 104o, 108o, 120o define an overlapping configuration (e.g., fully or partially overlapping). The lattice type (e.g., stochastic, unit cell, etc.), pore size, porosity, thickness, and struct size of the first, second, and third lattice structures 104o, 108o, 120o are customizable and are selected based on the desired roughness of the outer surface 110o of the device 100o.

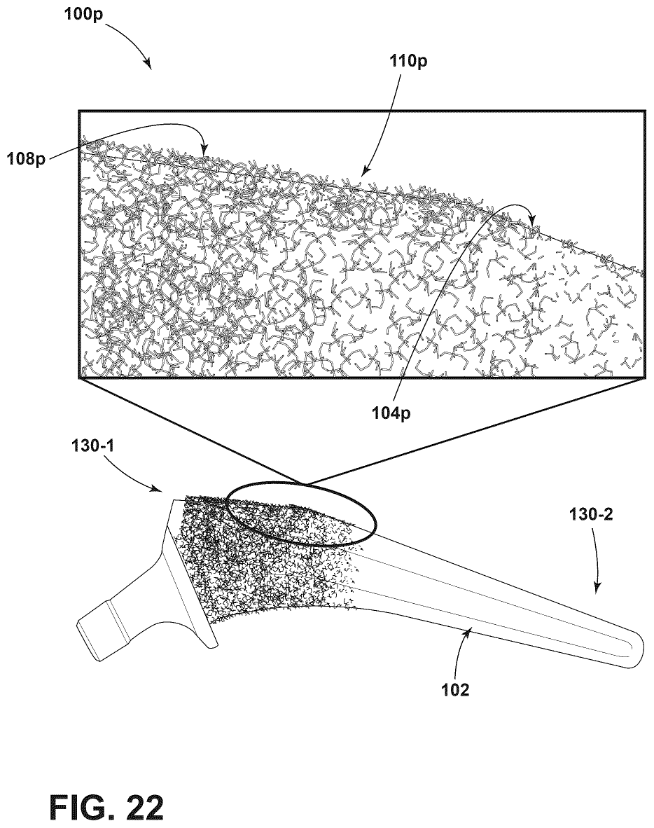

With reference to FIGS. 22-24, an example device 100p is shown. In view of the similarity in structure and function of the device 100p to the devices 100-100o, like reference numerals are used hereinafter and in the drawings to identify like components while like reference numerals containing letter extensions (e.g., “p”) are used to identify those components that have been modified.

The device 100p may comprise one or more of a variety of shapes, sizes, configurations, and/or materials. In various implementations, the device 100p is a hip stem implant. In various implementations, the device 100p includes a substrate 102, a first lattice structure 104p, and a second lattice structure 108p. The first lattice structure 104p may define a base body and the second lattice structure 108p may define a roughness body. The second lattice structure 108p may be intermixed with the first lattice structure 104p.

In various implementations, the thickness T1 of the first lattice structure 104p remains the same throughout the porous portion of the device 100p. The roughness of the outer surface 110p of the device 100p is the consequence of the difference between the thickness T1 of the first lattice structure 104p and the thickness T2 of the second lattice structure 108p. A first area of the outer surface 110p, closer to the distal end of the device 100p, has a thinner second lattice structure 108p with a smaller thickness difference with the first lattice structure 104p. The foregoing results in a smoother surface compared to a second area of the outer surface 110p closer to the neck region (e.g., the proximal end) of the device 100p. The second area has a thicker second lattice structure 108p that sticks out of the first lattice structure more and, therefore creating a rougher surface and facilitating a better grip into a bone of patient. The foregoing characteristics of the first and second lattice structures 104p, 108p provide the outer surface 110p of the device 100p with a graded (e.g., gradually increasing) roughness.

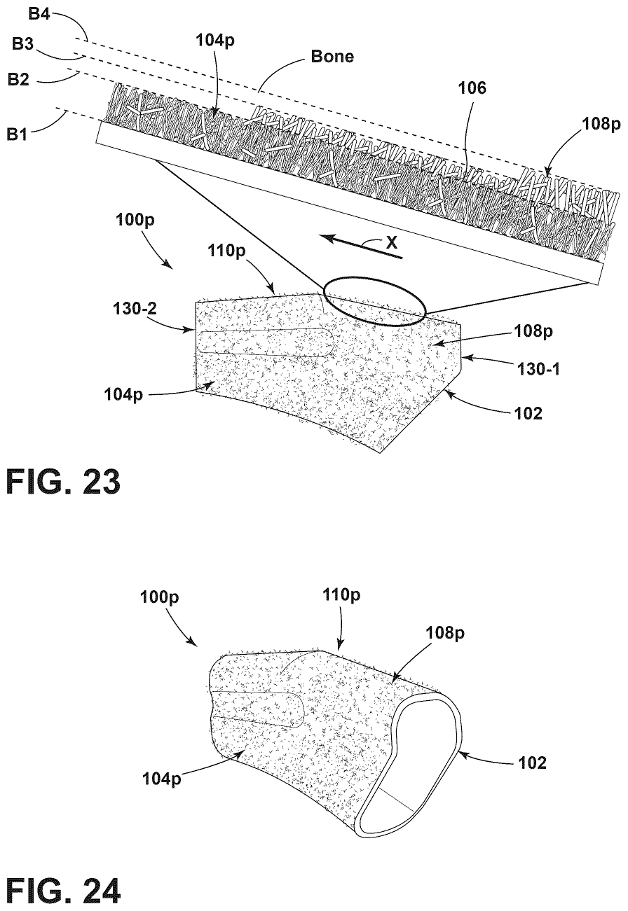

Referring now to FIG. 23, the first and second lattice structures 104, 108p may define a first boundary B1, a second boundary B2, a third boundary B3, and a fourth boundary B4, among others. The first boundary B1 may be disposed adjacent to the surface 106. The second boundary B2 may be disposed between the first and third boundaries B1, B3. The third boundary B3 may be disposed between the second and fourth boundaries B2, B4. The fourth boundary B4 may be disposed between the third boundary 3 and a bone of a recipient (e.g., a patient) of the device 100p.

The roughness of the boundaries B1-B4 may vary. For example, the third boundary B3 may have a greater roughness than the second boundary B2. The fourth boundary B4 may have a greater roughness than the third boundary B3. The foregoing enables the device 100p to have a varying interference fit with the bone, for example, when the device is inserted (e.g., in the X-direction) into the recipient. In other words, the distal end 130-1 of the device 100p may be rougher than a proximal end 130-2 of the device 100p.

In some examples, the struts of the first and second lattice structures 104p, 108p define the boundaries B1-B4. For example, the struts of the first lattice structure 104p may define the first and second boundaries B1, B2. The struts of the second lattice structure 108p may define the third and fourth boundaries B3, B4.

At least one of the boundaries B1-B4 may define a first roughness value proximate the distal end 130-1 and a second roughness value proximate the proximal end 130-2. The first roughness value may be greater than the second roughness value. The first roughness value may be less than the second roughness value. At least one of the boundaries B1-B4 may define a third roughness value between the distal end 130-1 and the proximal end 130-2. The third roughness value may be greater than the second roughness value. The third roughness value may be less than the first roughness value.

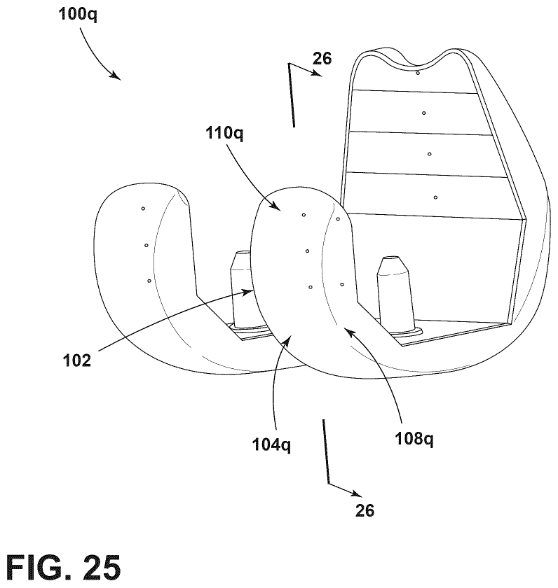

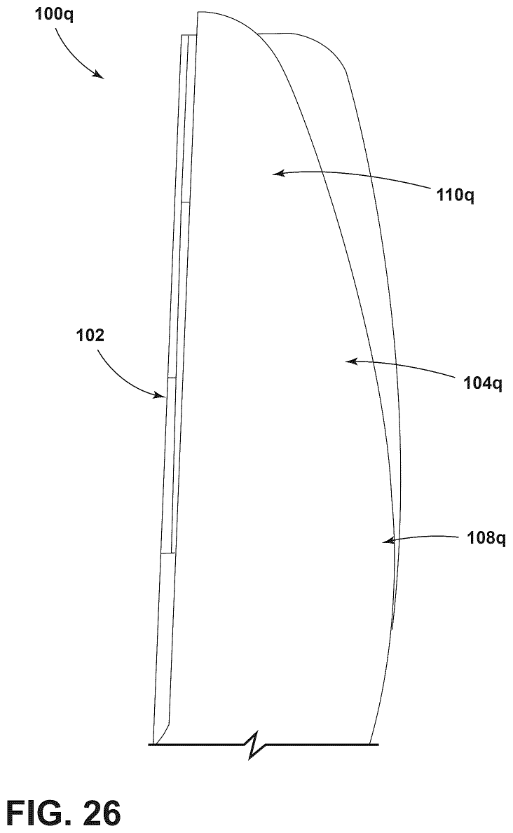

With reference to FIGS. 25 and 26, an example device 100q is shown. In view of the similarity in structure and function of the device 100q to the devices 100-100p, like reference numerals are used hereinafter and in the drawings to identify like components while like reference numerals containing letter extensions (e.g., “q”) are used to identify those components that have been modified.

The device 100q may comprise one or more of a variety of shapes, sizes, configurations, and/or materials. In various implementations, the device 100q is a femoral knee component. In various implementations, the device 100q includes a substrate 102, a first lattice structure 104q, and a second lattice structure 108q. The first lattice structure 104q may define a base body and the second lattice structure 108q may define a roughness body.

In various implementations, the thickness T2 of the second lattice structure 108q is constant, while the thickness T1 of the first lattice structure 104q is selectively reduced creating a bigger thickness difference between the lattice structures, and therefore creating a rougher outer surface 110q. An area of the outer surface 110q, closer to the anterior flange and the posterior condyles, has less difference between the thicknesses of the first and second lattice structures 104q, 108q resulting in smoother ease of insertion during surgery. The foregoing characteristics of the first and second lattice structures 104q, 108q provide the outer surface 110q of the device 100q with a graded (e.g., gradually increasing) roughness

In various implementations, a device (e.g., at least one of the devices 100-100q) may have a mechanical integrity pull strength of greater than 2900 psi (20 MPa) and a shear strength of greater than 2900 psi (20 MPa). The device may have abrasion resistance (ASTM F98) of less than 65 mg while having percent porosity in range of 35-70% and average pore sizes in the range 0.25 to 0.1 mm.

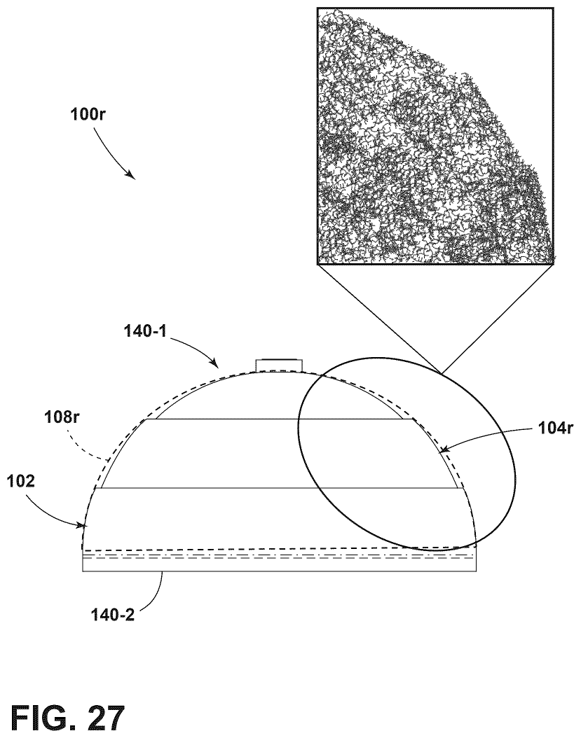

With reference to FIG. 27, an example device 100r is shown. In view of the similarity in structure and function of the device 100r to the devices 100-100q, like reference numerals are used hereinafter and in the drawings to identify like components while like reference numerals containing letter extensions (e.g., “r”) are used to identify those components that have been modified.

The device 100r may comprise one or more of a variety of shapes, sizes, configurations, and/or materials. In various implementations, the device 100r is an acetabular cup. In various implementations, the device 100r includes a substrate 102, a first lattice structure 104r, and a second lattice structure 108r. The first lattice structure 104r may define a base body and the second lattice structure 108r may define a roughness body. The thickness of the first lattice structure 104r may transition from thinner at a proximal end 140-1 of the device 100r to thicker towards a distal end 140-2. The thickness of the second lattice structure 108r may be constant from the proximal end 140-1 to the distal end 140-2. The foregoing provides the device 100r with a graded (e.g., gradually increasing) roughness over a constant overall thickness.

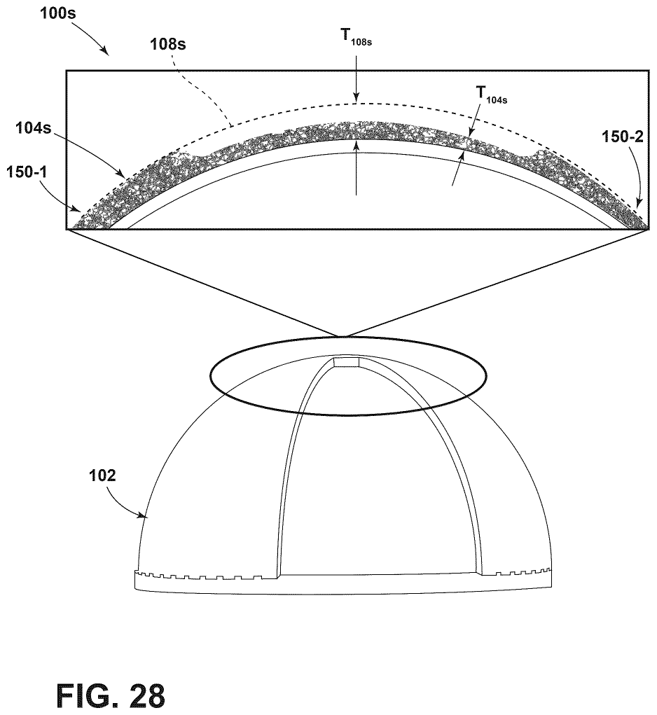

With reference to FIG. 28, an example device 100s is shown. In view of the similarity in structure and function of the device 100s to the devices 100-100r, like reference numerals are used hereinafter and in the drawings to identify like components while like reference numerals containing letter extensions (e.g., “s”) are used to identify those components that have been modified.

The device 100s may comprise one or more of a variety of shapes, sizes, configurations, and/or materials. In various implementations, the device 100s is an acetabular cup. In various implementations, the device 100s includes a substrate 102, a first lattice structure 104s, and a second lattice structure 108s. The first lattice structure 104s may define a base body and the second lattice structure 108s may define a roughness body. The thickness T_{104S} of the first lattice structure 104r may transition from thicker at first and second ends 150-1, 150-2 of the device 100s to thinner at a section(s) of the device 100s disposed between the first and second ends 150-1, 150-2. The thickness T_{108S} of the second lattice structure 108s may be constant from the first end 150-1 to the second end 150-2. The foregoing provides the device 100s with roughness that alternates between smooth and rough.

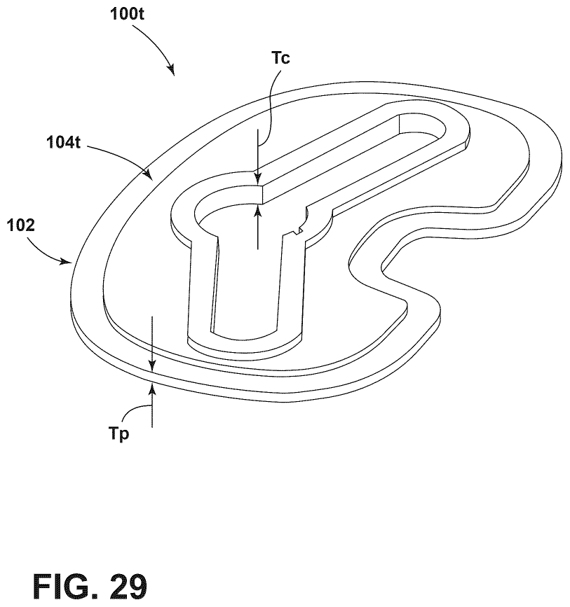

With reference to FIG. 29, an example device 100t is shown. In view of the similarity in structure and function of the device 100t to the devices 100-100s, like reference numerals are used hereinafter and in the drawings to identify like components while like reference numerals containing letter extensions (e.g., “t”) are used to identify those components that have been modified.

The device 100t may comprise one or more of a variety of shapes, sizes, configurations, and/or materials. In various implementations, the device 100t is a tibia base plate. In various implementations, the device 100t includes a substrate 102, a first lattice structure 104t, and a second lattice structure (not shown). The first lattice structure 104t may define a base body and the second lattice structure may define a roughness body. The thickness of the first lattice structure 104t may transition from thinner (e.g., Tp) on the periphery of the device 100t to thicker (e.g., Tc) toward the keel of the device 100t. The thickness of the second lattice structure may be constant. The foregoing enables the device 100t to be rougher proximate the periphery and smoother inwards from the periphery.

As illustrated in FIG. 2, a first lattice structure (e.g., first lattice structure 104-104t) may include a first plurality of struts 114-1 defining a first plurality of pores 116-1, and a second lattice structure (e.g., second lattice structure 108-108t) may include a second plurality of struts 114-2 defining a second plurality of pores 116-2. In some implementations, the size (e.g., length, width, depth, etc.) of the struts 114-1, 114-2 and/or pores 116-1, 116-2 varies such that the characteristics of the lattice structure 104-104t, 108-108t vary within each lattice structure and/or between the lattice structures. For example, the struts 114-1, 114-2 may define various cross-sectional shapes, such as a circle, a triangle, or a rectangle, among others.

In some implementations, the second lattice structure (e.g., second lattice structure 108-108t) may overlay the first lattice structure (e.g., first lattice structure 104-104t). Accordingly, the second lattice structure (e.g., second lattice structure 108-108t) may be referred to as an “overlay lattice.” In some implementations, the first lattice structure (e.g., first lattice structure 104-104t) may be intermingled (e.g., intertwined, interwoven, intermixed, etc.) with the second lattice structure (e.g., second lattice structure 108-108t). For example, as illustrated in FIG. 2, the struts 114-1 of the first lattice structure (e.g., first lattice structure 104-104t) may be interwoven with the struts 114-2 of the second lattice structure (e.g., second lattice structure 108-108t) such that the first and second lattice structures are disposed within the same volume V (see, e.g., FIG. 23).

A method of manufacturing a device (e.g., at least one of the devices 100-100t) may include forming a substrate (e.g., substrate 102). In some implementations, the substrate 102 may be formed by an additive manufacturing process such as electron beam melting (EBM), laser powder bed fusion (LPBF), binder jet (BJ), and stereolithography (SL), among others. It will be appreciated, however, that the substrate 102 may be formed using other methods (e.g., casting, molding, etc.) within the scope of the present disclosure. The method may further include forming a lattice structure (e.g., the lattice structures 104a-104t). The lattices structures 104a-104t may be attached to a pre-formed substrate 102 via techniques known in the art (e.g., sintering, using adhesives, diffusion bonding, etc.) In some implementations, the method includes integrally (e.g., monolithically) forming the lattice structure 104a-104t with the substrate 102 using an additive manufacturing process such as electron beam melting (EBM), laser powder bed fusion (LPBF), binder jet (BJ), and stereolithography (SL), among others. Other methods such as direct energy deposition (DED) may be used to apply lattices structures 104a-104t on previously-formed substrates 102, such that the lattice structures 104a-104t and substrate 102 are formed separately.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

Spatial and functional relationships between elements (for example, between modules, layers, etc.) are described using various terms, including “connected,” “engaged,” “coupled,” “adjacent,” “proximate,” “next to,” “on top of,” “above,” “below,” and “disposed.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present between the first and second elements. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The term “set” does not necessarily exclude the empty set—in other words, in some circumstances a “set” may have zero elements. The term “non-empty set” may be used to indicate exclusion of the empty set—in other words, a non-empty set will always have one or more elements. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR.

Various example embodiments of the invention are described in the following clauses

### Clause 1

A device comprising: a substrate; a first lattice structure coupled to the substrate and defining a first plurality of pores; and a second lattice structure coupled to at least one of the substrate or the first lattice structure and at least partially disposed within the first plurality of pores, the second lattice structure defining a second plurality of pores in fluid communication with the first plurality of pores.

### Clause 2

The device of clause 1 wherein: the first and second lattice structures are intermingled, the first and second lattice structures cover at least a portion of the outer surface of the substrate, and locations and characteristics of the first and second lattice structures correlate to a surface roughness of the device.

### Clause 3

The device of any of clauses 1 or 2 wherein the second lattice structure has at least one characteristic that is different than the first lattice structure.

### Clause 4

The device of clause 3 wherein the at least one characteristic includes a porosity, a pore size, a strut size, a lattice type, or a thickness.

### Clause 5

The device of any of clauses 1 through 4 wherein: the first lattice structure defines a first thickness, and the second lattice structure defines a second thickness.

### Clause 6

The device of clause 5 wherein the second thickness is greater than the first thickness.

### Clause 7

The device of any of clauses 5 or 6 wherein the second thickness is less than the first thickness.

### Clause 8

The device of any of clauses 1 through 7 wherein one of the first or second lattice structures defines a stochastic structure and the other one of the first or second lattice structures defines a unit cell structure.

### Clause 9

The device of any of clauses 1 through 8 wherein each of the first and second lattice structures define stochastic structures.

### Clause 10

The device of any of clauses 1 through 9 wherein each of the first and second lattice structures define unit cell structures.

### Clause 11

The device of any of clauses 1 through 10 wherein: the first lattice structure defines a first strut size, the second lattice structure defines a second strut size, and the first strut size is different than the second strut size.

### Clause 12

The device of any of clauses 1 through 11 wherein: one or more pores of the first plurality of pores define a first pore size, one or more pores of the second plurality of pores define a second pore size, and the first pore size is different than the second pore size.

### Clause 13

The device of any of clauses 1 through 12 wherein the first lattice structure defines a different pore size and a different porosity in comparison with the second lattice structure.

### Clause 14

The device of any of clauses 1 through 13 wherein the first lattice structure has a first roughness value, and the second lattice structure has a second roughness value.

### Clause 15

The device of any of clauses 1 through 14 wherein at least one of the first lattice structure or the second lattice structure defines a graded thickness.

### Clause 16

The device of any of clauses 1 through 15 wherein increasing at least one of a first thickness of the first lattice structure or a second thickness of the second lattice structure increases a roughness value of an outer boundary of the device.

### Clause 17

The device of any of clauses 1 through 16 wherein at least one of the first lattice structure or the second lattice structure defines a wedge-shaped outer boundary.

### Clause 18

The device of any of clauses 1 through 17 further comprising: a third lattice structure coupled to at least one of the substrate, the first lattice structure, or the second lattice structure.

### Clause 19

The device of any of clauses 1 through 18 wherein the device is an implant.

### Clause 20

The device of clause 19 wherein the implant includes a hip stem having a proximal end and a distal end opposite the proximal end, and wherein at least one of the first lattice structure or the second lattice structure defines a first roughness at the proximal end and a second roughness at the distal end, and wherein the first roughness is greater than the second roughness.

### Clause 21

The device of clause 20 wherein the implant includes a hip stem having a lateral side and a medial side opposite the lateral side, and wherein at least one of the first lattice structure or the second lattice structure defines a first roughness at the lateral side and a second roughness at the medial side, and wherein the second roughness is greater than the first roughness.

### Clause 22

The device of clause 21 wherein the hip stem includes a proximal end and a distal end opposite the proximal end, and wherein at least one of the first lattice structure or the second lattice structure defines a third roughness at the proximal end and a fourth roughness at the distal end, and wherein the third roughness is greater than the fourth roughness.

### Clause 23

The device of any of clauses 1 through 22 wherein the device is a femoral knee component.

### Clause 24

A method of manufacturing a device, the method comprising: forming a first lattice structure on a substrate, the first lattice structure defining a first plurality of pores; and forming a second lattice structure within the first plurality of pores, the second lattice structure defining a second plurality of pores in fluid communication with the first plurality of pores.

### Clause 25

The method of clause 24 further comprising forming a second lattice structure on at least one of the substrate or the first lattice structure, wherein the second lattice structure defines an additional porous structure.

### Clause 26

The method of clause 25 wherein the second lattice structure has at least one characteristic that is different than the first lattice structure.

### Clause 27

The method of clause 26 wherein the at least one characteristic includes a porosity, a pore size, a strut size, a lattice type, or a thickness.

### Clause 28

The method of any of clauses 24 through 27 wherein: the first lattice structure defines a first thickness, and the second lattice structure defines a second thickness.

### Clause 29

The method of clause 28 wherein the second thickness is greater than the first thickness.

### Clause 30

The method of any of clauses 28 through 29 wherein the second thickness is less than the first thickness.

### Clause 31

The method of any of clauses 24 through 30 wherein one of the first or second lattice structures defines a stochastic structure and the other one of the first or second lattice structures defines a unit cell structure.

### Clause 32

A device comprising: a substrate, a first lattice structure coupled to the substrate and including a first plurality of struts defining a first outermost boundary offset from the substrate; and a second lattice structure coupled to at least one of the substrate or the first lattice structure and including a second plurality of struts defining a second outermost boundary offset from the first outermost boundary.

### Clause 33

An implant comprising: a substrate including a proximal end and a distal end opposite the proximal end; a first lattice structure coupled to the substrate between the proximal end and the distal end; a second lattice structure intermingled with the first lattice structure between the proximal end and the distal end, at least one of the first lattice structure or the second lattice structure including a plurality of struts defining an outermost boundary offset from the substrate, wherein the outermost boundary defines a first roughness value at the distal end and a second roughness value that is less than the first roughness value at the proximal end.

### Clause 34

The implant of clause 33 wherein the outermost boundary defines a third roughness value between the proximal end and the distal end, the third roughness value being greater than the second roughness value and less than the first roughness value.

### Clause 35

An implant produced by a process comprising: providing a biocompatible substrate; subjecting the substrate to a first additive manufacturing process to form a first lattice structure defining a first plurality of pores; and subjecting the substrate to a second additive manufacturing process to form a second lattice structure defining a second plurality of pores in fluid communication with the first plurality of pores.

1. A device comprising:
a substrate;
a first lattice structure coupled to the substrate and defining a first plurality of pores; and
a second lattice structure coupled to at least one of the substrate or the first lattice structure and at least partially disposed within the first plurality of pores, the second lattice structure defining a second plurality of pores in fluid communication with the first plurality of pores.

2. The device of claim 1 wherein:
the first and second lattice structures are intermingled,
the first and second lattice structures cover at least a portion of an outer surface of the substrate, and
locations and characteristics of the first and second lattice structures correlate to a surface roughness of the device.

3. The device of claim 1 wherein the second lattice structure has at least one characteristic that is different than the first lattice structure.

4. The device of claim 3 wherein the at least one characteristic includes a porosity, a pore size, a strut size, a lattice type, or a thickness.

5. The device of claim 1 wherein:
the first lattice structure defines a first thickness, and
the second lattice structure defines a second thickness.

6. The device of claim 5 wherein the second thickness is greater than the first thickness.

7. The device of claim 5 wherein the second thickness is less than the first thickness.

8. The device of claim 1 wherein one of the first or second lattice structures form a stochastic structure and the other one of the first or second lattice structures form a unit cell structure.

9. The device of claim 1 wherein each of the first and second lattice structures form stochastic structures.

10. The device of claim 1 wherein each of the first and second lattice structures form unit cell structures.

11. The device of claim 1 wherein:
the first lattice structure defines a first strut size,
the second lattice structure defines a second strut size, and
the first strut size is different than the second strut size.

12. The device of claim 1 wherein:
one or more pores of the first plurality of pores define a first pore size,
one or more pores of the second plurality of pores define a second pore size, and
the first pore size is different than the second pore size.

13. The device of claim 1 wherein the first lattice structure defines a different pore size and a different porosity in comparison with the second lattice structure.

14. The device of claim 1 wherein the first lattice structure has a first roughness value, and the second lattice structure has a second roughness value.

15. The device of claim 1 wherein at least one of the first lattice structure or the second lattice structure defines a graded thickness.

16. The device of claim 1 wherein increasing at least one of a first thickness of the first lattice structure or a second thickness of the second lattice structure increases a roughness value of an outer boundary of the device.

17. The device of claim 1 wherein at least one of the first lattice structure or the second lattice structure defines a wedge-shaped outer boundary.

18. The device of claim 1 further comprising:
a third lattice structure coupled to at least one of the substrate, the first lattice structure, or the second lattice structure.

19. The device of claim 1 wherein the device is an implant.

20. The device of claim 19 wherein the implant includes a hip stem having a proximal end and a distal end opposite the proximal end, and wherein at least one of the first lattice structure or the second lattice structure defines a first roughness at the proximal end and a second roughness at the distal end, and wherein the first roughness is greater than the second roughness.

21. The device of claim 20 wherein the implant includes a hip stem having a lateral side and a medial side opposite the lateral side, and wherein at least one of the first lattice structure or the second lattice structure defines a first roughness at the lateral side and a second roughness at the medial side, and wherein the second roughness is greater than the first roughness.

22. The device of claim 21 wherein the hip stem includes a proximal end and a distal end opposite the proximal end, and wherein at least one of the first lattice structure or the second lattice structure defines a third roughness at the proximal end and a fourth roughness at the distal end, and wherein the third roughness is greater than the fourth roughness.

23. The device of claim 1 wherein the device is a femoral knee component.

24. A method of manufacturing a device, the method comprising:
forming a first lattice structure on a substrate, the first lattice structure defining a first plurality of pores; and
forming a second lattice structure within the first plurality of pores, the second lattice structure defining a second plurality of pores in fluid communication with the first plurality of pores.

25. The method of claim 24 further comprising forming a second lattice structure on at least one of the substrate or the first lattice structure, wherein the second lattice structure defines an additional porous structure.

26. The method of claim 25 wherein the second lattice structure has at least one characteristic that is different than the first lattice structure.

27. The method of claim 26 wherein the at least one characteristic includes a porosity, a pore size, a strut size, a lattice type, or a thickness.

28. The method of claim 24 wherein:
the first lattice structure defines a first thickness, and
the second lattice structure defines a second thickness.

29. The method of claim 28 wherein the second thickness is greater than the first thickness.

30. The method of claim 28 wherein the second thickness is less than the first thickness.

31. The method of claim 24 wherein one of the first or second lattice structures defines a stochastic structure and the other one of the first or second lattice structures defines a unit cell structure.

32. A device comprising:
a substrate;
a first lattice structure coupled to the substrate and including a first plurality of struts defining a first outermost boundary offset from the substrate; and
a second lattice structure coupled to at least one of the substrate or the first lattice structure and including a second plurality of struts defining a second outermost boundary offset from the first outermost boundary.

33. An implant comprising:
a substrate including a proximal end and a distal end opposite the proximal end;
a first lattice structure coupled to the substrate between the proximal end and the distal end; and
a second lattice structure intermingled with the first lattice structure between the proximal end and the distal end, at least one of the first lattice structure or the second lattice structure including a plurality of struts defining an outermost boundary offset from the substrate, wherein the outermost boundary defines a first roughness value at the distal end and a second roughness value that is less than the first roughness value at the proximal end.

34. The implant of claim 33 wherein the outermost boundary defines a third roughness value between the proximal end and the distal end, the third roughness value being greater than the second roughness value and less than the first roughness value.

35. An implant produced by a process comprising:
providing a biocompatible substrate;
subjecting the substrate to a first additive manufacturing process to form a first lattice structure defining a first plurality of pores; and subjecting the substrate to a second additive manufacturing process to form a second lattice structure defining a second plurality of pores in fluid communication with the first plurality of pores.
